(19) United States
(12) Patent Application Publication
(10) Pub. No.: US 2021/0007836 A1
(43) Pub. Date: Jan. 14, 2021

(54) Stress Urinary Incontinence Treatment Devices And Methods

(71) Applicant: Caldera Medical, Inc., Agoura Hills, CA (US)

(72) Inventor: Ronald D. Adams, Holliston, MA (US)

(73) Assignee: Caldera Medical, Inc., Agoura Hills, CA (US)

(21) Appl. No.: 16/921,752

(22) Filed: Jul. 6, 2020

Related U.S. Application Data
(60) Provisional application No. 62/871,470, filed on Jul. 8, 2019.

(51) Int. Cl. A61F 2/00; A61M 39/02; A61M 39/24

(52) U.S. Cl. CPC A61F 2/004; A61M 39/0208; A61M 39/24; A61F 2250/0031; A61F 2220/0075; A61F 2250/0013; A61F 2210/0004; A61F 2220/0016

(57) ABSTRACT

Apparatus and methods are provided to position a device at adjacent tissues near a urethra to overcome the problem of urine leakage during physical movement or activity by changing a contour of a urethra. The devices may include mechanisms such as an inflatable pouch or a pillow anchored to the adjacent tissues of a urethra by an anchoring hook. The inflation of the pouch by an external fluid flexes the pouch towards a urethra and changes the contour of the urethra. The devices may also or alternatively include one or more tissue grasping T-fasteners having forward-facing and reverse-facing barbs to anchor the tissues adjacent to a urethra and bend the contour of the urethra. The device may also include actuators, clips or spring jacks having inflatable bands. The device may include a cap, one-way valves and a combination of a plug and cap to block urethral ostium to temporarily block urine flow from a bladder.

### RELATED APPLICATIONS

This application claims priority to U.S. Provisional Application Ser. No. 62/871,470 filed Jul. 8, 2019 entitled SUI Treatment Devices And Methods, which is hereby incorporated herein by reference in its entirety.

### FIELD OF THE INVENTION

The present invention describes embodiments of a novel systems that may be used to inhibit the loss of urine during physical activity or movement.

### BACKGROUND OF THE INVENTION

Stress Urinary Incontinence (SUI) involves the inability of the urethral/vesical system to prevent the leakage of urine when physical movement or activity, such as, coughing, sneezing, running or heavy lifting puts pressure (stress) on the bladder. Stress incontinence occurs when the muscles and other tissues that support the bladder (pelvic floor muscles) and the muscles that regulate the release of urine (urinary sphincter) weaken.

The bladder expands as it fills with urine. Normally, valve-like muscles in the urethra, the short tube that carries urine out of your body, stay closed as the bladder expands, preventing urine leakage until you reach a bathroom. But when those muscles weaken, anything that exerts force on the abdominal and pelvic muscles, such as, sneezing, bending over, lifting, laughing hard, for instance can put pressure on your bladder and cause urine leakage.

In women, pelvic floor muscles and urinary sphincter may lose strength because of distension of tissues or nerve damage during childbirth, resulting in stress incontinence. This stress incontinence may begin soon after delivery or occur years later.

The treatments for SUI include behavioral therapies, medications, devices and surgery. Behavioral therapies include pelvic floor muscle exercises (Kegal exercises), changing fluid consumption habits, healthy lifestyle habit changes such as weight reduction or discontinuation of smoking and bladder training. While some success with behavioral therapies has been observed, they require activity and sustained efforts on the part of the patient for success.

Medical therapies such as antidepressants have been investigated but symptoms quickly return once medication is discontinued and these medications are often associated with nausea. Devices have offered most successful treatments for SUI and these include vaginal pessaries and urethral inserts. These devices have demonstrated some success with mitigating the SUI but do require the patient to manipulate them manually. The urethra inserts are not intended for daily use so periodic replacement is necessary.

Several surgical options are available including injectable bulking agents, retro pubic Culpo-suspension and sling procedures. Injection of bulking agents has offered short term symptomatic relief, but additional injections are required to maintain their efficacy. Sacral colpopexy procedures are a common surgical approach where the apex of the vagina is attached to the sacrum via a piece of medical mesh. These procedures are conducted in the OR under general anesthesia and involve a number of co-morbidities that can significantly impact patient quality of life. Sling procedures have become the standard of care due to their less invasive nature, simpler training requirements and overall efficacy. However, a number of class action lawsuits have been filed and settled against manufacturers of mesh devices for the pelvic floor area.

There is therefore a need for alternatives that can support the urethra to maintain urinary continence with a simple, easy to implement means that is adjustable.

### OBJECTS AND SUMMARY OF THE INVENTION

The present application overcomes the problem of Stress Urinary Incontinence by changing the contour of the urethra thereby inhibiting urine leakage during physical movement or activity.

In some embodiments of the present invention, an apparatus is used to change a contour of the urethra. The apparatus comprising a body having a first configuration and a second configuration, an anchoring mechanism usable to anchor the body at a location adjacent to a urethra. In the first configuration of the body, the body allows urine to flow through said urethra and in the second configuration of the body, the body changes shape such that said urethra is displaced from a natural position to a compressed position between said body and an anatomic structure of a patient, thereby reducing urine flow through said urethra.

According to some embodiments, the present invention uses an apparatus to modulate urine flow from the urethra. The apparatus comprises a tissue grasping device having a distal grasping end and a proximal grasping end connected by a filament. The distal grasping end anchors tissues adjacent to the urethra and the proximal grasping end anchors tissues adjacent to a deployment site, thereby compressing said urethra and modulating urine flow through said urethra.

In some embodiments, the present application describes a method of inhibiting urine flow through the urethra. The method involves positioning an inflatable device at a desired location adjacent to the urethra in a first configuration. The device changes shape to a second configuration when it is inflated. The inflation of the device thereby compresses the urethra against adjacent tissue and inhibits the urine flow through said urethra.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1A is a side view of an embodiment of a pillow system with a pillow, anchoring hook, suture and flexible tube of the present invention.

FIG. 1B is a top view of an embodiment of a pillow constructed by laminating two or more thin sheets of film materials.

FIG. 2A is a side view of two thin sheets of materials used for lamination to form an embodiment of a pillow of the invention.

FIG. 2B is a plan view of the pillow system showed in FIGS. 1A and 1B.

FIG. 3 is a depiction of a step of an embodiment of a method for installing an embodiment of a pillow system adjacent to the urethra.

FIG. 4 is a depiction of a step of an embodiment of a method for installing an embodiment of a pillow system adjacent to the urethra.

FIG. 5 is a side view of an embodiment of a pillow system of the invention installed adjacent to a urethra.

FIG. 6 shows an embodiment of an inflation method of the invention.

FIG. 7 shows an embodiment of an adequately inflated pillow adjacent to a urethra after the inflation procedure of FIG. 6.

FIG. 8 is a side view of an embodiment of a seal of the invention.

FIG. 9 is a side view of an embodiment of a seal of the invention.

FIG. 10 is a side view of an embodiment of a seal of the invention.

FIG. 11 is a side view of an embodiment of a seal of the invention.

FIG. 12 is a side view of an embodiment of a seal of the invention.

FIG. 13 is a plan view of an embodiment of a pillow system of the invention having a pillow with saw-toothed surface.

FIG. 14 is a plan view of an embodiment of a pillow system of the invention having a pillow with holes on a surface.

FIG. 15 is a plan view of an embodiment of a pillow system of the invention having a pillow with block patterns on a surface.

FIG. 16 is a depiction of a step of an embodiment of a method of the invention for removing a pillow system from adjacent region of a urethra.

FIG. 17 is a side view of an embodiment of a pillow system of the invention with an additional reservoir installed below a skin surface.

FIG. 18 is a depiction of a step of an embodiment of a method of the invention in which a pillow system having a pillow as a containment pouch is filled with an active agent.

FIG. 19 is a top view of a containment pouch of the invention having an impermeable membrane and capillary paths on a surface.

FIG. 20 is a top view of a containment pouch of the invention filled with growth hormone.

FIG. 21 is a top view of an embodiment of a T-fastener of the invention.

FIG. 22 is a depiction of a step of an embodiment of a method of the invention for installing a T-fastener between the vaginal wall and adjacent tissue of the urethra.

FIG. 23 is a side view of a urethral bend achieved by an embodiment of a T-fastener of the invention.

FIG. 24 is a top view of an embodiment of the invention in which multiple T-fasteners are tied between the vaginal wall and adjacent tissue of the urethra.

FIG. 25 is a top view of an embodiment of a T-fastener of the invention being used to create an anterior kink of a urethra.

FIG. 26 is a side view of an embodiment of a T-fastener of the invention being used to create an anterior kink of a urethra.

FIG. 27 is a side view of an embodiment of a plurality of T-fasteners of the invention being used to create an anterior kink of a urethra.

FIG. 28 is a top view of an embodiment of a T-fastener of the invention being used to create an anterior kink of a urethra.

FIG. 29 is a side view of an embodiment of a T-fastener of the invention being used to create an anterior kink of a urethra.

FIG. 30 is a side view of an embodiment of a plurality of T-fasteners of the invention being used to create an anterior kink of a urethra.

FIG. 31 is an axial cross section of a urethra being compressed by an embodiment of a T-fastener of the invention.

FIG. 32 is a longitudinal cross section of a urethra being compressed by an embodiment of a T-fastener of the invention.

FIG. 33 is a longitudinal cross section of a urethra being compressed by a plurality of T-fasteners of the invention.

FIG. 34 a top view of an embodiment of a bi-directional T-fastener of the invention having barbed ends.

FIG. 35 is a perspective view of an embodiment of a delivery device of a bi-directional T-fastener of the invention having barbed ends.

FIG. 36 is a perspective view of an embodiment of a delivery tube having a bi-directional T-fastener positioned inside of the invention.

FIG. 37 is a depiction of a step of an embodiment of a method of the invention for installation of a bi-directional T-fastener between obturator foramen and uterofacial tissue or vaginal wall.

FIG. 38 is a depiction of a step of an embodiment of a method of the invention for installation of a bi-directional T-fastener between obturator foramen and uterofacial tissue or vaginal wall.

FIG. 39 is a top view of two T-fasteners of the invention anchored between obturator foramen and uterofacial tissue or vaginal wall from both sides.

FIG. 40 is a front view of two T-fasteners of the invention anchored between obturator foramen and uterofacial tissue or vaginal wall from both sides.

FIG. 41 is a depiction of a step of an embodiment of a method of the invention for installation of a bi-directional T-fastener between the Cooper's Ligament and vaginal wall.

FIG. 42 is a depiction of a step of an embodiment of a method of the invention for installation of a bi-directional T-fastener in which a tip of a delivery tube reaches the vaginal wall.

FIG. 43 is a depiction of a step of an embodiment of a method of the invention for installation of a bi-directional T-fastener in which an outer delivery tube is removed.

FIG. 44 is a front view of two T-fasteners of the invention anchored between the Cooper's Ligament and vaginal wall.

FIG. 45 is a top view of a T-fastener of the invention anchored between the Cooper's Ligament and vaginal wall by reverse-facings barbs and forward-facing barbs.

FIG. 46 is a front view of a female pelvic floor illuminated with a light source of the invention.

FIG. 47 is a side view of an embodiment of a light source of the invention.

FIG. 48 is a side view of an embodiment of a delivery device of a bi-directional T-fastener having an LED light at a distal tip of the invention.

FIG. 49 is a top view of an embodiment of a delivery device having a digital camera, monitor and an irrigation port of the invention.

FIG. 50A is a side view of an embodiment of an anchoring system having a mesh attached to an anchoring hook of the invention.

FIG. 50B is top view of an embodiment of an anchoring system including a mesh having barbs on a surface attached to an anchoring hook of the invention.

FIG. 51 is a top view of an embodiment of piezoelectric actuators positioned adjacent to a urethra in deflated states of the invention.

FIG. 52 is top view of an embodiment of piezoelectric actuators positioned adjacent to a urethra in inflated states of the invention.

FIG. 53 a side view of an embodiment of a piezoelectric actuator base having a ratcheting surface and a stripe attached to the base in a deflated state of the invention.

FIG. 54 a side view of an embodiment of a piezoelectric actuator base having a ratcheting surface and a stripe attached to the base in an inflated state of the invention.

FIG. 55 a side view of an embodiment of a spring jack having a flexible band in a deflated state of the invention.

FIG. 56 is a depiction of an embodiment of a step of the invention to activate a spring jack to inflate a flexible band.

FIG. 57A is a front view of an embodiment of a spring jack having a flexible band positioned adjacent to a urethra in a deflated state of the invention.

FIG. 57B is a front view of an embodiment of a spring jack having a flexible band positioned adjacent to a urethra in an inflated state of the invention.

FIG. 58A is a side view of an embodiment of a flexing clip having a ratcheting track and a flexing member attached to the ratcheting track in a deflated state of the invention.

FIG. 58B is a side view of an embodiment of a flexing clip having a ratcheting track and a flexing member attached to the ratcheting track in an inflated state of the invention.

FIG. 59 is a side view of an embodiment of a delivery tool to activate the flexing clip showed in FIGS. 58A and 58B of the invention.

FIG. 60A is a side view of an embodiment of a urethral plug to reduce temporarily the urine flow of the invention.

FIG. 60B shows a position of insertion of a urethral plug inside a urethral ostium of the invention.

FIG. 61 is a side view of an embodiment of a plug having ribbed surface of the invention.

FIG. 62 is a side view of an embodiment of a duckbill-valve to reduce the urine flow temporarily from a bladder of the invention.

FIG. 63 is a side view of an embodiment of a dome-valve to reduce the urine flow temporarily from a bladder of the invention.

FIG. 64 is a depiction of an embodiment of a step of the invention to open a duckbill-valve.

FIG. 65 is a depiction of an embodiment of a step of the invention to open a dome-valve.

FIG. 66 is a top view of an embodiment of a duckbill-valve and a dome-valve stabilized at desired locations by mesh structures of the invention.

FIG. 67 is a depiction of an embodiment of a step of a method of the invention to inhibit the flow of urine by a magnetic valve housing comprising a receiver and a sphere.

FIG. 68 is a depiction of an embodiment of a step of a method of the invention to release the flow of urine through a magnetic valve housing comprising a receiver and a sphere.

FIG. 69 is a depiction of an embodiment of a step of a method of the invention to inhibit the flow of urine by a magnetic valve housing comprising a receiver and a rod.

FIG. 70 is a depiction of an embodiment of a step of a method of the invention to release the flow of urine through a magnetic valve housing comprising a receiver and a rod.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

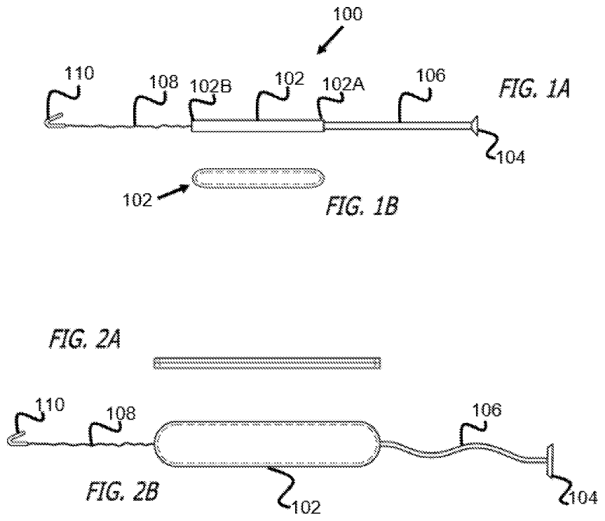

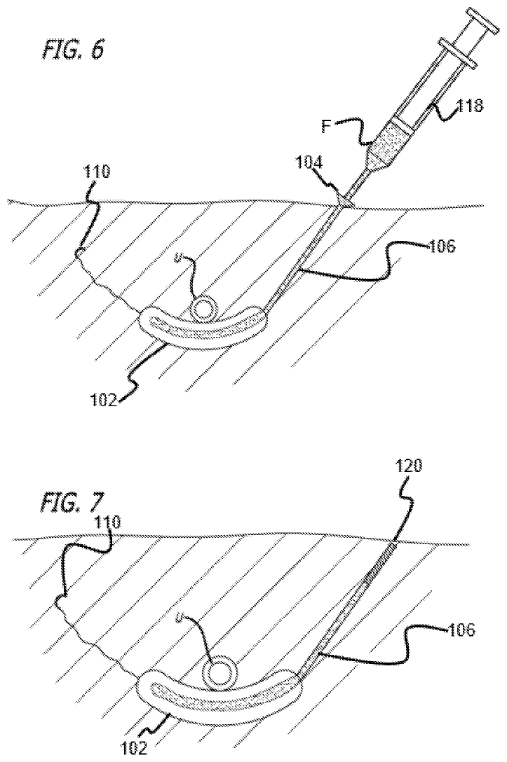

Referring initially to FIG. 1A, an adjustable pillow system 100 of the present invention generally includes a pillow or bladder 102 having a proximal end 102A and a distal end 102B. A flexible tube 106 is connected to the proximal end 102A of the pillow 102. The flexible tube 106 further includes an access port 104 at the proximal end which provides an entry portal for the delivery of a fluid inside the pillow 102. The access port 104 and connected flexible tube 106 provide a pathway for adjusting the inflation and deflation of the pillow 102. In some embodiments, the diameter of the flexible tube 106 may be about 0.25 OD. As the pillow 102 expands with the delivery of a fluid, it puts pressure on the tissues surrounding the urethra U (FIG. 6) creating a change in configuration or contour that will inhibit the flow of urine from the bladder. A strand of material such as a suture 108 is connected to the distal end 102B of the pillow 102. In some embodiments, the suture 108 is seam-welded to bond with the distal end 102B of the pillow 102. An anchoring hook 110 is further attached to an end of the suture 108 opposite to the end which is connected to the distal end 102B of the pillow 102. The anchoring hook 110 may be utilized in the delivery of the pillow 102 to the treatment site adjacent to the urethra U.

As can be seen in FIG. 1B, the pillow or bladder 102 may be created by bonding together two thin sheets of materials, for example, the non-limiting materials of the pillow or bladder 102 are polypropylene, polyethylene, nylon, PVC coated fabrics etc. FIG. 2A further shows that any of the two combinations of thin sheets materials may be laminated together to form pillow 102. The non-limiting examples of laminations of two thin sheets together may comprise radio frequency welding, heat welding, lamination using adhesives or solvent bonding.

Referring to an enlarged view of the adjustable pillow system 100 in FIG. 2B, the pillow or bladder 102 may be formed by seem welding any of the two combinations of polypropylene, polyethylene, nylon, PVC coated fabrics thin sheets at their respective seams. In some embodiments, it may be preferable to have one of the two thin sheets of materials be constructed of a stiffer material such that a bias on the direction of movement of the less stiff material of inflated sheet is created. In some embodiments, it may be necessary for the bottom layer of the two layers to be stiffer than the top layer to facilitate an expansion of the top layer that is more unidirectional towards the surrounding tissues of the urethra U.

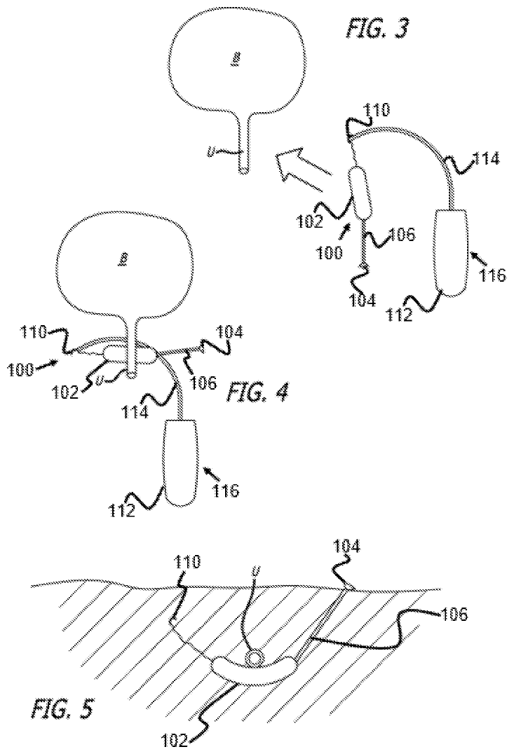

FIG. 3 shows a step to load the adjustable pillow system 100 of the present invention onto an introducer system 116. The introducer system 116 comprises a handle 112 and an angulated needle 114 attached to the handle 112. The angulated needle 114 may be used to engage the anchoring hook 110 of the adjustable pillow system 100. FIG. 4 shows that once the adjustable pillow system 100 is anchored with the angulated needle 114 through the anchoring hook 110, the adjustable pillow system 100 is inserted into the transurethral tissues through an abdominal incision to place the pillow section 102 adjacent to the urethra U.

FIG. 5 shows that the adjustable pillow system 100 of the present invention is placed at the desired location adjacent to the urethra U with the anchoring hook 110 engaging the surrounding tissues such that the pillow system 100 may not be moved once it is placed at the desired location. FIG. 5 further shows that the access port 104 is positioned at or above the skin level to enable access for the delivery of a fluid F inside the pillow 102.

Referring now to FIG. 6, once the adjustable pillow system 100 of the present invention is properly installed at the desired location, the pillow 102 may be inflated with fluid F delivered using a syringe or other fluid dispensing device 118 through the access port 104. The pressure of the pillow 102 may be adjusted by varying the amount of fluid F. The adjustment of pillow pressure may be made while the patient coughs to exert pressure on the bladder. Once the pillow 102 is inflated to a desired level, the expansion of the pillow flexes the surrounding tissues of the urethra U to create a flow resistance of urine through the bladder.

FIG. 7 further shows that once the proper inflation of the pillow 102 has been achieved, the flexible tube 106 can be blocked by inserting a plug 120. Alternatively, the tube 106 could be clamped, tied, clipped, etc. The plug could also be replaced with a valve or it could be tied off with suture and the access port 104 removed. Non-limiting examples of closure mechanisms are shown in the Figures and described below.

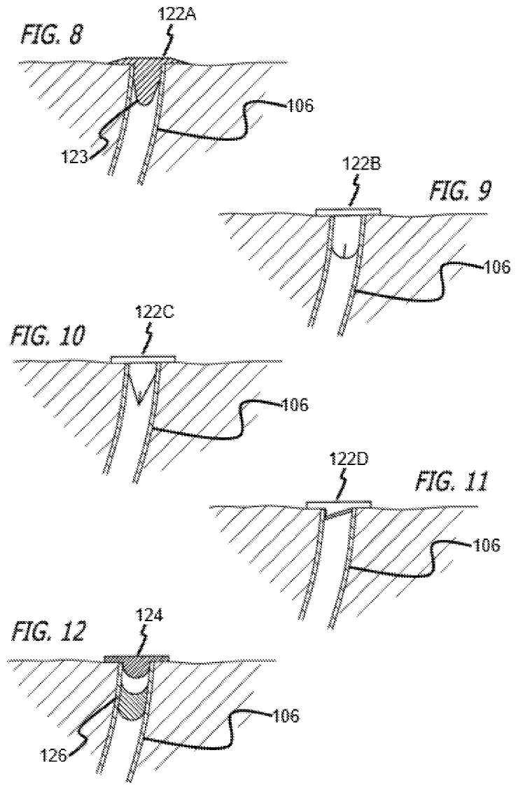

For example, FIGS. 8-12 disclose various sealing embodiments to seal the flexible tube 106 that would maintain adjusted pillow pressure to keep the pillow 102 expanded. FIG. 8 shows an embodiment that includes a removable cap 122A with a shaped protruding member 123 that fits into the inflation tube 106 to seal it. The removable cap 122A remains at the skin surface such that it could be removed to allow access to the flexible tube 106. FIGS. 9-11 show different embodiments of valves (122B, 122C and 122D) to seal the inflation tube 106. FIG. 9 shows a one-way flow valve, for example, a dome valve 122B and FIG. 10 shows another example of a one-way flow valve, a duckbill valve 122C. FIG. 11 shows an example of a flapper valve 122D to seal the flexible tube 106.

FIG. 12 shows an alternative embodiment to seal the flexible tube 106 with a combination of a plug 124 and an absorbable cap 126. In some embodiments, the plug 124 may be made from an implantable, non-bio absorbing material such as, polypropylene to create a durable seal and the cap 126 is made of polyglycolic acid (PGA) or polylactic Acid (PLA). Without limiting the materials to make the plug 124 and cap 126 to polypropylene, PGA or PLA, it should be known that the plug 124 and cap 126 may be made by any suitable implantable, non-bio absorbing materials.

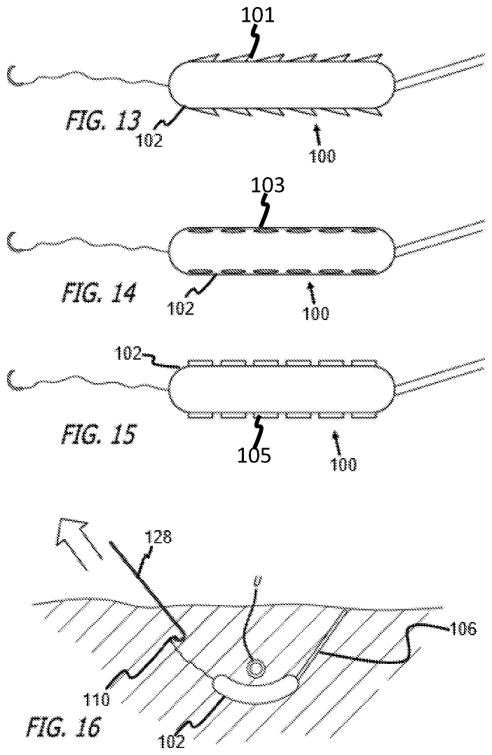

Referring now to FIGS. 13-15, these figures show various embodiments to stabilize the pillow system 100 at the desired location adjacent to the urethra U. The non-limiting examples of these embodiments include a saw-toothed edge 101 on the periphery of the pillow 102 (FIG. 13). Other embodiments include patterns such as holes 103 in the interior surface of the pillow 102 (FIG. 14) or a block pattern 105 on the exterior surface of the pillow (FIG. 15). These undulated surfaces of the pillow 102 create enough friction with the surrounding tissues and stabilize the pillow 102 at the desired location adjacent to the urethra U.

FIG. 16 shows a step to remove the pillow assembly 100 from the surrounding tissues of the urethra U. In some embodiments, the removal of the pillow assembly 100 involves non-limiting examples of a hook 128, a Kelly clamp or similar grasping devices (not shown in figures). The first step of removal involves making an abdominal incision to insert the angulated portion of the hook 128, Kelly clamp or similar grasping devices at the desired location. The second step involves capturing the anchoring hook 110 of the pillow assembly 100 by the angulated portion of the hook 128, Kelly clamp or similar grasping devices. Once the angulated portion of the hook 128 engages the anchoring hook 110, a gentle pressure is applied to remove the pillow assembly 100 out of the pelvic floor. In some embodiments, the anchoring hook 110 of the pillow assembly 100 may be coated with a radiopaque material, such as, barium sulfate infused polymeric material for easy detection of the anchoring hook 110 for engaging with the hook 128 with the surrounding tissues adjacent to the urethra U.

In some embodiments, in the port configuration of the pillow assembly 100, the access port 104 of the flexible tube 106 is removed and a syringe could be used to remove the fluid from the pillow 102 to deflate the pillow 102. The pillow assembly 100 may then be removed by engaging the anchoring hook 110 using hook 128 and applying necessary force to displace it from the surrounding tissues. In some embodiments, when the flexible tube 106 of the pillow assembly 100 is plugged by the cap 122A or different embodiments of one-way valves 122B, 122C and 122D or a combination of a plug 124 and an absorbable cap 126, as described in FIGS. 8-12, an ultrasound detection may be used to locate the pillow 102 of the pillow assembly 100 inside the body. The removable cap 122A, one-way valves 122B, 122C and 122D or a plug 124 and an absorbable cap 126 may then be pierced with a syringe (not shown in the figures) to extract the fluid and deflate the pillow 102 before the pillow assembly 100 is removed from the desired location.

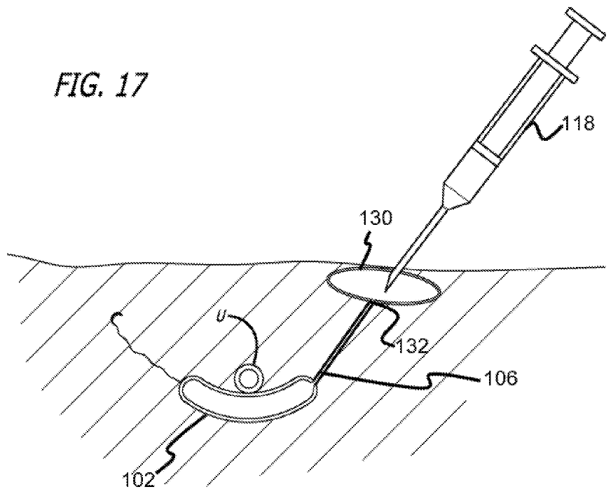

FIG. 17 shows an alternative embodiment of the pillow assembly 100 in which a reservoir 130 is attached to the flexible tube 106. The reservoir 130 may be positioned just below the skin surface and it provides fluid filled volume which may be adjusted for the distension of the pillow 102. This embodiment further includes a one-way valve 132 at the connection point to the flexible tube 106. The one-way valve 132 offers the patient the option to adjust the pressure of the pillow 102 by pushing the reservoir with a finger so that more fluid can be diverted through the one-way valve 132 into the pillow 102 to distend it and increase the urine flow resistance of the urethra U. In some embodiments, the reservoir 130 may be positioned outside the body of the patient such that the manipulations of the fluid pressure inside the pillow 102 would be easier. In some embodiments, a syringe may be used to add or extract fluid from the reservoir 130 to control the urethral pressure by inflation or deflation of the pillow 102.

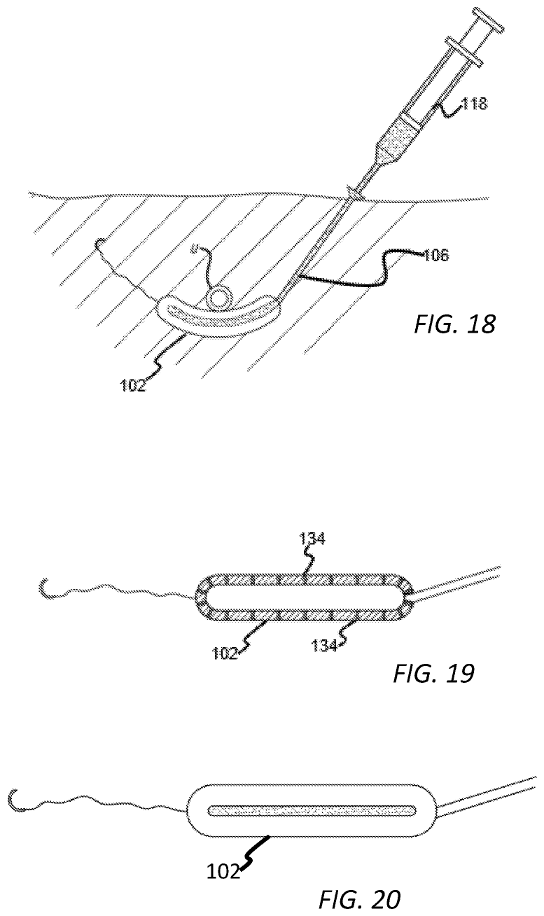

Referring now to FIGS. 18 and 19 of the present invention, these figures show another embodiment in which the pillow 102 of the pillow assembly 100 is being used as a containment pouch constructed of semi-permeable or impermeable membrane. The semipermeable or impermeable containment pillow 102 may be filled with an active agent, such as a growth hormone or platelet rich plasma (PRP) or other growth agents, injected from a syringe 118 into the containment pillow 102 through the flexible tube 106. The active agent slowly leaks out through the semipermeable or impermeable membrane of the containment pillow 102. In some embodiments, the semi-permeable membrane of the containment pillow 102 may be made of a combination of polymeric materials, such as a combination of polyamide and polysulfone or similar materials. In some embodiments, when the containment pillow 102 comprises impermeable membrane, capillary paths 134 may be needed on the surface of the containment pillow 102 to enable the growth hormone or platelet rich plasma to leak out over time. When the pillow assembly 100 is placed at the desired location adjacent to the urethra U, the active agent may be leached out through semipermeable membrane or capillary paths 134 of the containment pillow 102 and may create a longer lasting stimulation of the surrounding tissues of the urethra U.

In this embodiment, the containment pillow 102 would mimic a PICC (percutaneous inserted central catheter) line for the periodic delivery of the growth agents.

The containment pillow 102 may be constructed of two sheets of materials in which one or both materials are semipermeable membranes made of a combination of polymeric materials, such as polyamide, polysulfone or similar materials. In some embodiments, the leached-out active agents of the growth hormone or platelet rich plasma from the containment pillow 102 may promote regrowth of the damaged tissue surrounding the urethra U and consequently, the pressure from the regrown surrounding tissues would also apply pressure to the containment pillow 102 to extrude more of the active agents into the surround tissues, which is ultimately beneficial for inhibiting the urine loss from the bladder. In some embodiments, the containment pillow 102 may include leakage ports 134 for the dispersion of the active agent into the surrounding tissues of the urethra U when the containment pillow 102 is made of impermeable membrane.

The pillow assembly 100 comprising containment pillow 102 described in FIGS. 18-19 is beneficial over current practice which involves an injection of growth hormones or platelet rich plasma in a single patient visit. The single injection produces a short-term benefit, whereas a longer sustained release of these agents through pillow assembly 100 in FIGS. 18-19 may possibly produce a more sustained benefit.

FIG. 20 shows, the containment pillow 102 of the pillow assembly 100 may be coated with a human growth hormone such as, somatotropin or similar growth hormones to promote the ingrowth of tissues around the urethral lumen to stabilize the pillow assembly 100.

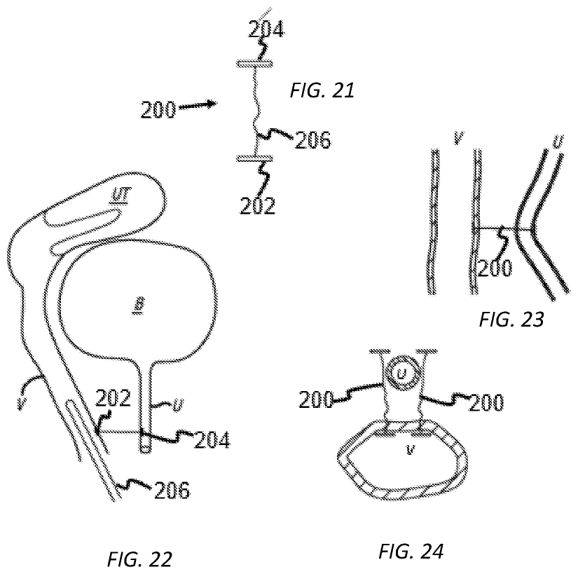

FIG. 21 shows an embodiment of a T-fastener 200 to treat Stress Urinary Incontinence. A T-fastener 200 is an adjustable fastener used to compress the transurethral tissues and tighten the musculature surrounding the urethra U in a manner that will create an increase in urine flow resistance to eliminate the loss of urine during physical activity. A T-fastener 200 comprises a first side 202 and a second side 204 connected by a suture 206 or similar materials.

As shown in FIG. 22, during an installation of the T-fastener 200, the T-fastener 200 may be loaded first on a delivery device 208, such as, a delivery tube and the delivery device 208 is inserted into the vagina V. Once the delivery device 208 is positioned at a desired location inside the vagina V, the T-fastener 200 may be released from the delivery device 208 such that the first side 202 of the T-fastener 200 remains anchored inside the vagina V and the second side 204 of the T-fastener 200 engages the transurethral tissues surrounding the urethra U to compress and tighten the urethra by creating a bend (FIG. 23) of the lumen of the urethra U. The compressed tissue will reshape the contour of the urethral lumen and create a more tortuous pathway for urine flow and thereby eliminate bladder leakage. In some embodiments, it may take multiple T-fasteners, as can be seen in FIG. 24, to adequately compress the tissues to create a “kink” or bend of the urethra U in a manner that creates enough resistance to the urine flow to mitigate the leakage.

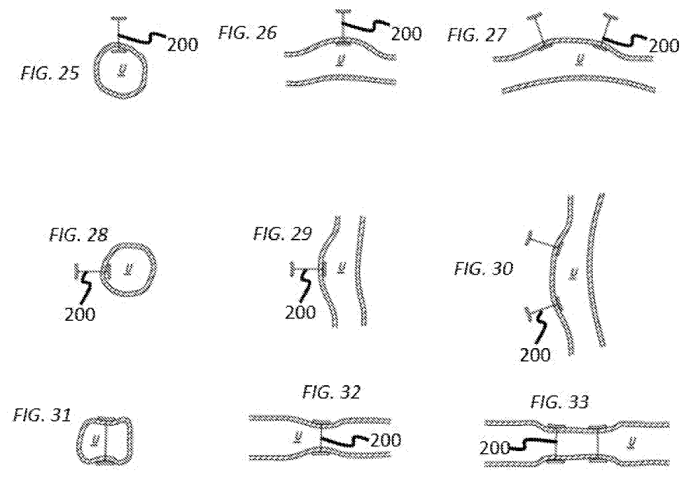

FIGS. 25-33 show several examples of intra-urethral approaches to modify the urethral geometry where T-fasteners 200 are utilized to create flow obstruction from within the lumen of the urethra U. As can be seen in FIGS. 25-27, placement of one or more T-fasteners 200 into the anterior space above the urethra U may produce an anterior kink. Similarly, FIGS. 28-30 show a kink or bend is produced by flexing the urethra either left or right side from its midline plane by intra-urethral placement of one or more T-fasteners 200 on the lateral margins of the urethra U. FIGS. 31-33 show a third approach to compress the lumen of the urethra U by placing one or more T-fasteners 200 across the cross section of the urethra U such that the one or more t-fasteners 200 traverse the lumen to bring its walls into a compressed to constrict or reduce the lumen cross section.

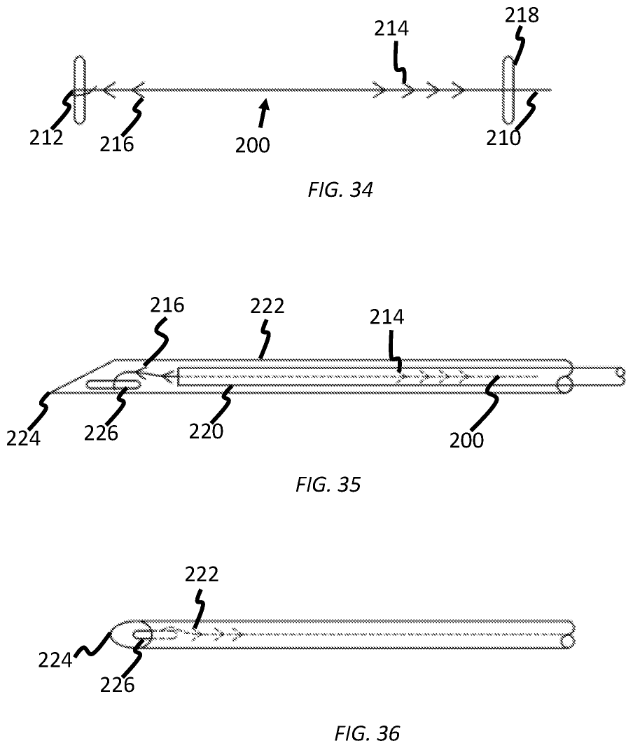

FIG. 34 show another embodiment of a T fastener 200, namely a bi-directional T-fastener 200 comprising a suture filament 207 with skived proximal and distal ends 210 and 212. The bi-directional T-fastener 200 further comprises forward facing barbs 216 and rearward facing barbs 214 positioned near the distal 212 and proximal 210 skived ends. In some embodiments, depending upon the strength of the tissues, it may be necessary to incorporate a bar or bolster 218 to increase the capacity of the bi-directional T-fastener 200 to maintain its position when installed.

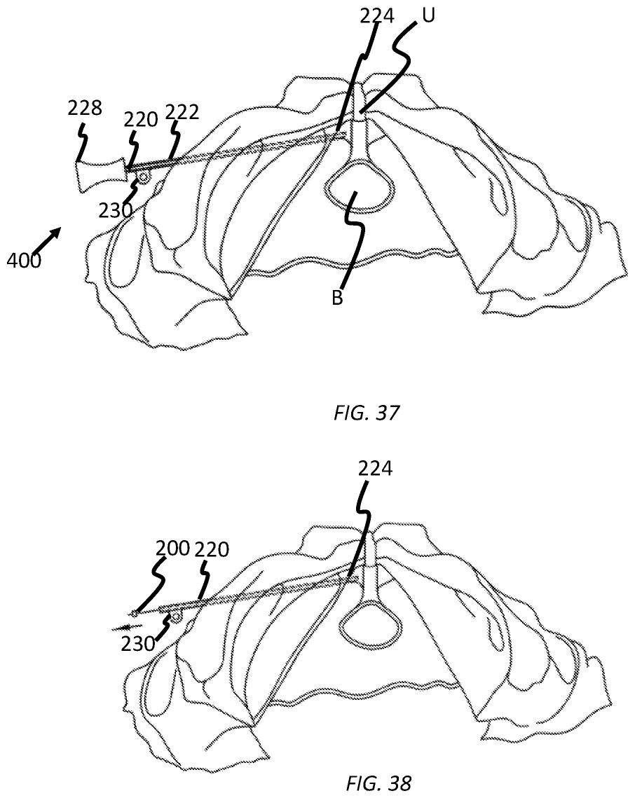

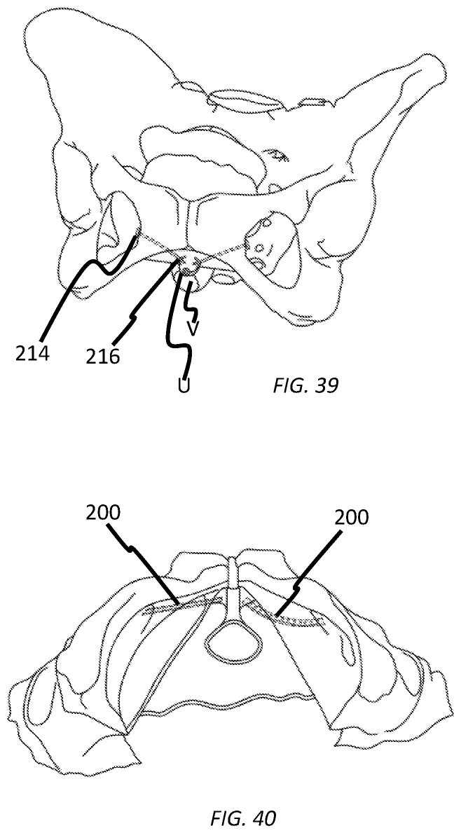

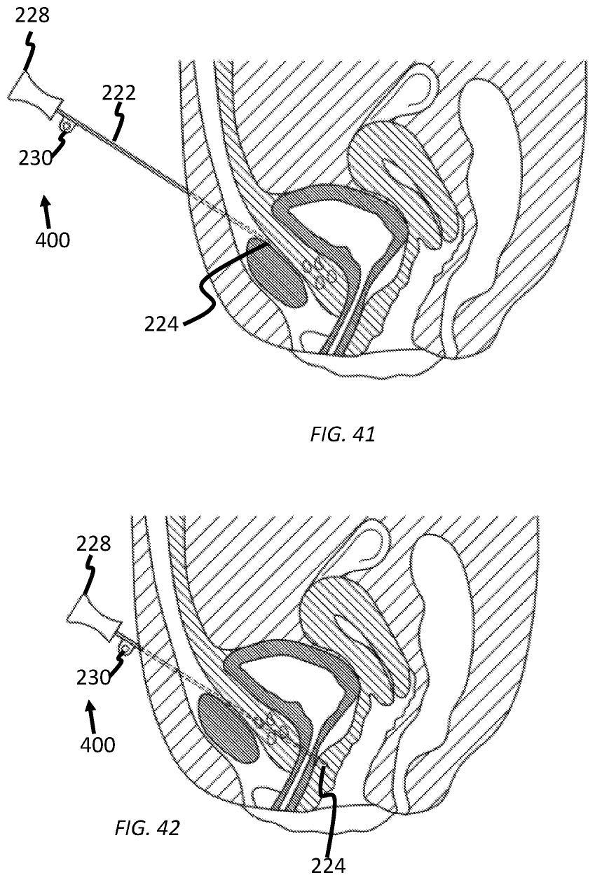

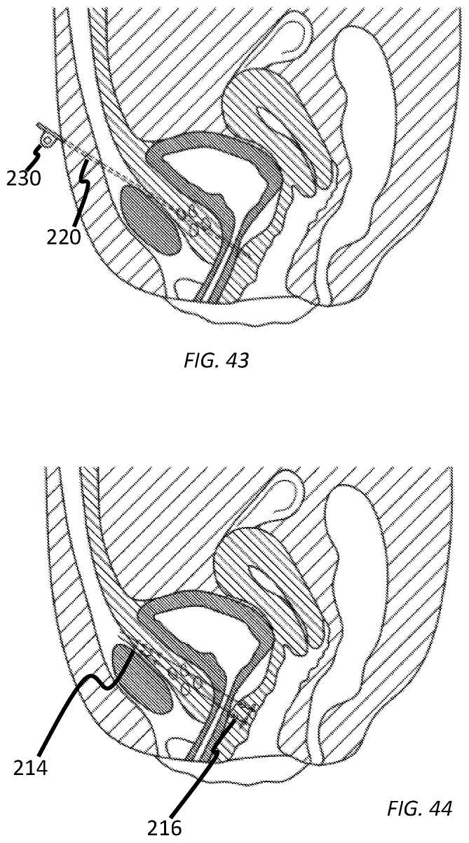

Referring now to FIGS. 35 and 36, it can be seen that the delivery system for the bi-directional T-fastener 200 includes an outer tube 222 and an inner tube 220. During the delivery inside the body, the bi-directional T-fastener 200 is housed inside the inner tube 220 and the inner tube 220 with the bi-directional T-fastener 200 is further housed inside the outer tube 222, as can be seen in FIG. 35. The outer tube 222 includes a piercing tip 224 and a deployment slot 226 (FIG. 36). The piercing tip 224 enables the outer tube 222 to pierce the skin surface such that the tube assembly may be inserted through various tissue layers and structures inside the body. Once the tube assembly reaches the desired position inside the vaginal wall, the bi-directional T-fastener 200 may be expelled out of the deployment slot 226 of the protective outer tube 222 to anchor the forward facing barbs 216 at the appropriate tissues of the vaginal wall.

FIG. 37 shows the anatomical elements of the female pelvis. The bladder (B) and urethra (U) are shown in the center of the drawing. FIG. 37 further shows a delivery device 400 of the tube assembly. In this figure, the outer tube 222 is connected to a first bigger handle 228 and the inner tube 220 is connected to a second smaller handle 230. The delivery device 400 may be advanced through the obturator foramen (OF) and the piercing tip 224 of the outer tube 222 is inserted into the uterofacial tissue or vaginal wall. FIG. 38 shows that once the piercing tip 224 of the outer tube 222 is properly reached near the subject tissues, the outer tube bigger handle 228 is retracted relative to the inner tube smaller handle 230 and the counter traction of the inner tube 220 results in the release of the distal end 212 of the bi-directional T-fastener 200 into the uterofacial tissue or vaginal wall through the deployment slot 226 of the outer tube 222 to engage the tissues at this desired location. Once the outer tube 222 is completely removed, the distal end 212 of the bi-directional T-fastener 200 is completely released to anchor into the uterofacial tissue or vaginal wall. Finally, a slight tug on the proximal end 210 of the bi-directional T-fastener 200 will engage the forward-facing barbs 216 into the surrounding tissues.

FIG. 39 shows that the inner tube 220 is also removed exposing the reverse facing barbs 214. As additional force is used to retract the bi-directional T-fastener 200, the reverse facing barbs 214 will be engaged with the obturator foramen (OF). If necessary, more traction force may be applied by sliding the bolster 218 over the reverse facing barbs 214.

FIG. 40 is an enlarged view of FIG. 39 showing two bi-directional T-fasteners 200 are anchored between the obturator foramen (OF) and the uterofacial tissue or vaginal wall.

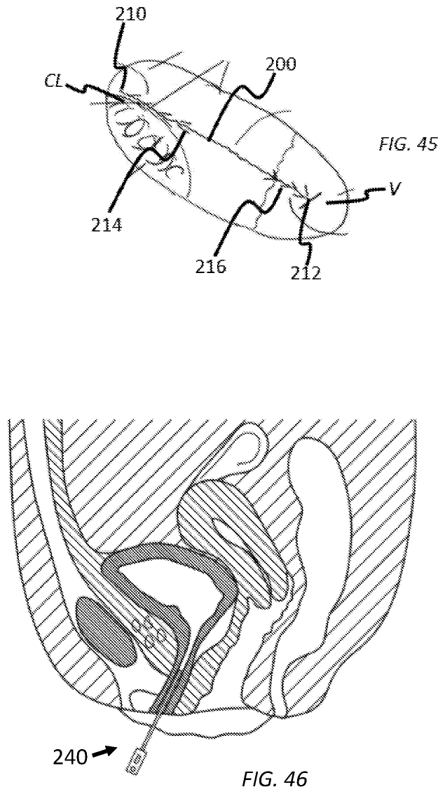

Referring to FIGS. 41-45, these figures show a second installation procedure of the bi-directional T-fastener 200 where Cooper's Ligament CL is utilized as the proximal anchoring location and the Prevesical Space of Retzius R is used as the distal anchoring location for engaging the uterofacial tissues or vaginal wall by the bi-directional T-fastener 200. FIG. 41 shows the delivery device 400 having the outer tube 222 is connected to a first bigger handle 228 and the inner tube (not shown in this figure) is connected to a second smaller handle 230. As shown in FIG. 36, the delivery device 400 in FIG. 41 also includes the outer tube 222 having a piercing tip 224 and a deployment slot (not shown in FIG. 41). During the installation procedure, an incision is made to the abdomen to enter the delivery device 400 into the abdominal cavity and the region of the Cooper's Ligament CL is pieced by the piercing tip of the outer tube 222. Next, the delivery device 400 may be advanced distally through the Prevesical Space of Retzius R and into the vaginal wall V, as can be seen in FIG. 42. FIG. 43 shows that once the piercing tip 224 of the delivery device 400 reaches the desired location distally, the outer tube bigger handle 228 is retracted relative to the inner tube smaller handle 230 and the counter traction of the inner tube results in the release of the forward-facing barbs 216 (FIGS. 44, 45) of the distal end of the bi-directional T-fastener 200 into the vaginal wall V to anchor the tissues into the vaginal wall. FIG. 44 shows that at the last step of the installation, the inner tube 220 is also removed exposing the reverse facing barbs 214. As additional force is used to retract the bi-directional T-fastener 200, the reverse facing barbs 214 will be released and engaged with the tissues around the Cooper's Ligament CL region of the pelvic floor.

FIG. 45 clearly shows that the forward-facing barbs 216 near the distal T-end 212 and the rearward facing barbs 214 near the proximal T-end 210 of the T-fastener 200 are anchored into the tissues of the vaginal wall V and the Cooper's Ligament CL, respectively.

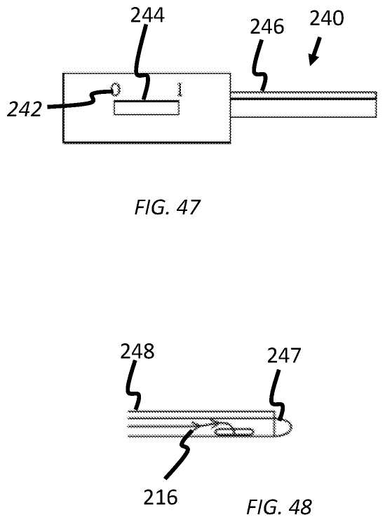

In some embodiments, a light source 240 may be used for easy visualization of the pelvic floor during the installation of the bi-directional T-fastener 200 (FIG. 46). The light source 240 may be inserted into the urethra U for illumination of the pelvic floor. The light source 240 comprises battery 242, power switch 244 and series of LED light sticks 246 (FIG. 47). The LED's may be color coded to indicate desired anchoring locations as described above.

FIG. 48 shows that in some embodiments, the distal tip of the delivery device 400 has an LED 247 located to illuminate its passage into the body. In this embodiment, the light source 240 is powered by a thin, flat cable 248 runs from the handle to the LED along the device shaft to deliver power to the light source 240.

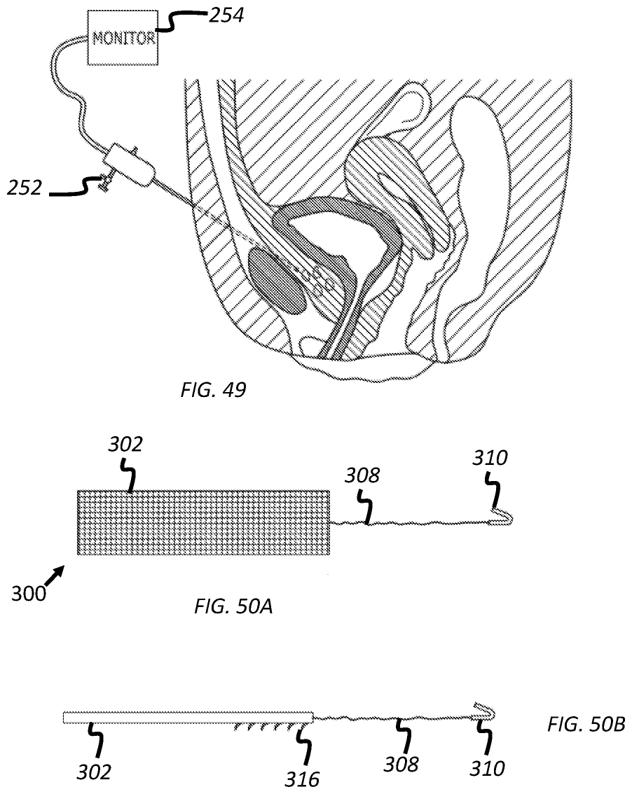

FIG. 49 shows another embodiment in which the installation procedure of the bi-directional T-fastener 200 includes a digital camera and a LED light source along with an irrigation port 252. The irrigation port 252 provides a means for hydro-dissection of the tissue planes in a gentle, safe manner. The digital camera is connected to a video monitor 254 to provide images to guide the placement of the bi-directional t-fastener 200.

FIG. 50A shows another embodiment of an anchoring system 300 where a mesh 302 is modified to add skives or barbs 316 to one of its surfaces. The mesh is attached to a suture or ligament 308 at one end and the other end of the suture 308 is attached to an anchoring hook 310. The mesh anchoring system 300 of the present invention is inserted into the tissues and is placed at the desired location adjacent to the urethra U with the anchoring hook 110 engaging the surrounding tissues such that the mesh anchoring system 300 may not be moved once it is placed at the desired location. The barbs 316 to one of the surfaces of the mesh 302 may further strengthen the anchoring of the mesh system 300 at the desired location (FIG. 50B). In some embodiments, a bidirectional mesh may be created where the distal attachment barbs are used to connect the mesh to the midline anchoring point (vaginal wall or uterofacial ligaments) and the proximal barbs are used to engage the obturator foremen or Cooper's Ligament (not shown in figures).

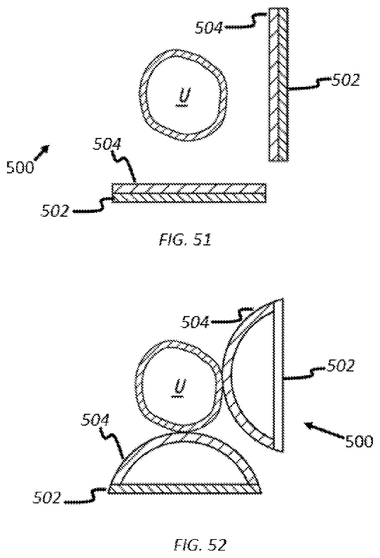

Referring to FIGS. 51-52, these figures show an embodiment to bend the contour of the urethra U to reduce the urine flow from the bladder. This embodiment involves two miniature piezoelectric actuators 500 comprising non-flexing bases 502 and flexing stripes 504 on top of the non-flexing bases 502. The piezoelectric actuators 500 may be placed adjacent to the urethra U and activated by using an electrical power source to stimulate the flexing of the stripes 504. When the stripes 504 flex, the tissues adjacent to the urethra U produce a kink or flexure of the urethra as necessary to bend the contour of the urethra U and reduce the urine flow from the bladder. Since the piezoelectric materials may pose a biocompatibility risk, it may be necessary to encapsulate the piezoelectric actuators 500 into membranes or similar protective housings.

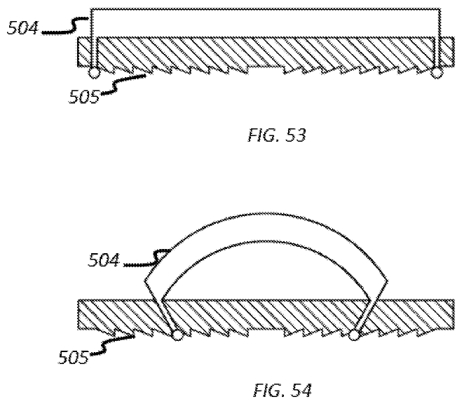

FIGS. 53 and 54 show that a ratcheting surface 505 of the base 502 may be necessary to stabilize the base 502 at the desired location when the stripes 504 flex.

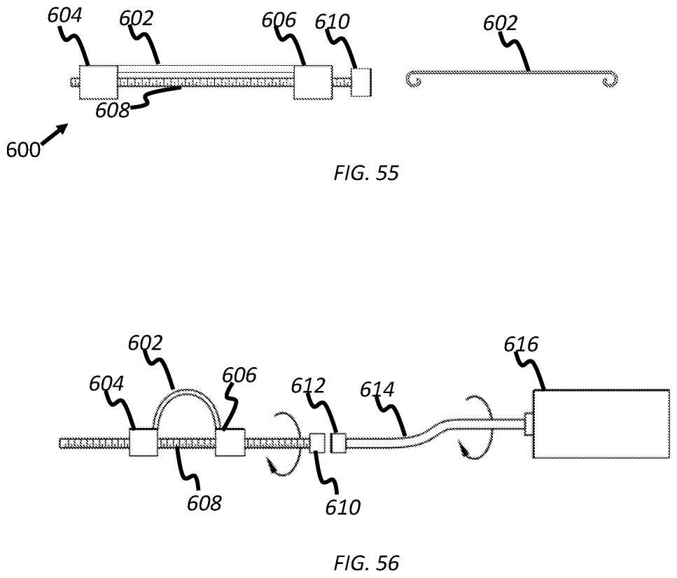

Referring to FIGS. 55-57B, these figures show an embodiment to bend the contour of the urethra U to reduce the urine flow from the bladder. This embodiment includes a spring jack 600 comprising a flexible band 602 attached to threaded collars 604, 606 that are mounted onto a threaded rod 608 having a handle 610. FIG. 56 further shows that a motor 616 with a drive system is attached with the handle 610 of the spring jack 600 through a coupling member 612 and an electric wire 614. As the drive system is electrically activated, the rotation of the coupling member 612 rotates the threaded rod 608 and bring the two threaded collars 604, 606 together. Consequently, as the threaded collars 604, 606 close the gap, the compression of the flexible band 602 causes it to deflect into a semi-circular orientation. The deflected flexible band 602 will put pressure onto the adjacent tissues to bend the contour of the urethra U and reduce the urine flow from the bladder. In some embodiments, the kink or bend of the urethra may be adjusted by the clockwise or anticlockwise rotation of the threaded rod 608.

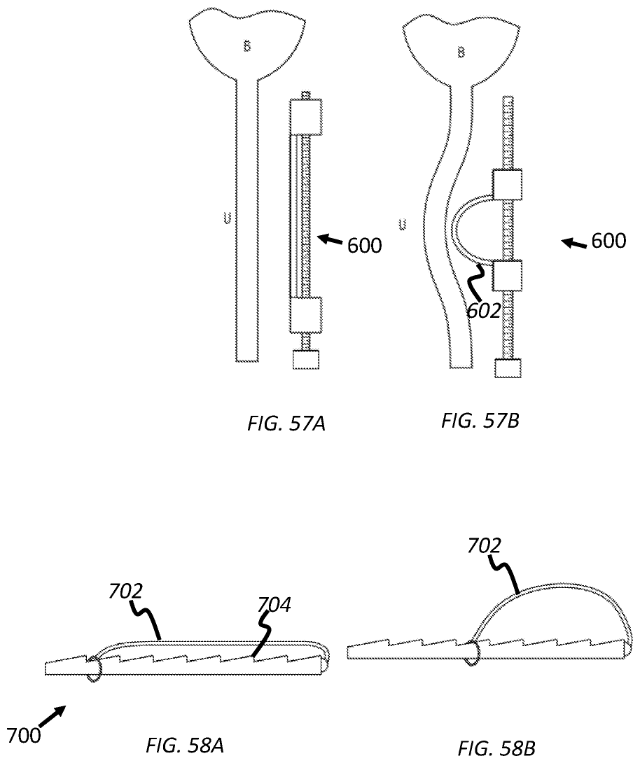

FIG. 57A shows that the flexible band 602 of the spring jack 600 is positioned near the urethra U in non-flexing state and FIG. 57B shows that the flexible band 602 of the spring jack 600 flexes to change the contour of the urethra U.

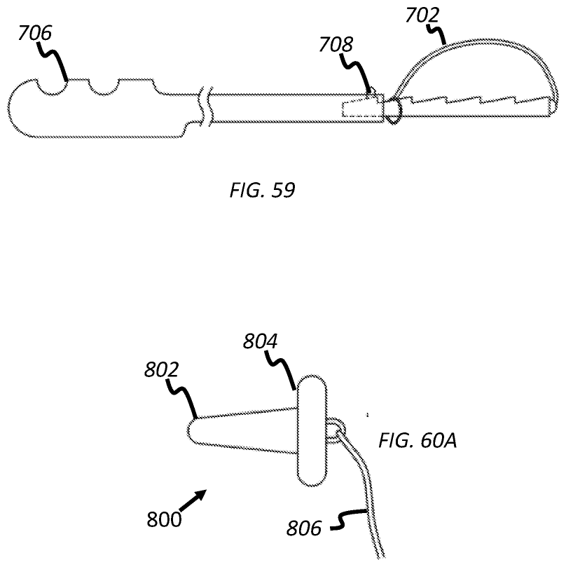

Referring now to FIGS. 58A-59 of the present invention, these figures show an embodiment to bend the contour of the urethra U to reduce the urine flow from the bladder by using a flexing clip 700. The flexing clip 700 comprises a flexing member 702 and a ratcheting track 704 (FIG. 58A). A delivery and pushing tool 706 may be used to insert and activate the flexing clip 700 (FIG. 59). The pushing tool further includes a ball detent 708 and release mechanism (not shown) for the ball detent 708 that allows the pushing tool 706 to hold the flexing clip 700 during insertion and to provide counter traction on the ratcheted ratcheting track 704. As the pushing tool 706 is advanced to drive the flexible member 702, the flexible member 702 flexes to a semi-circular shape to put pressure onto the adjacent tissues to bend the contour of the urethra U and reduce the urine flow from the bladder (FIG. 58B).

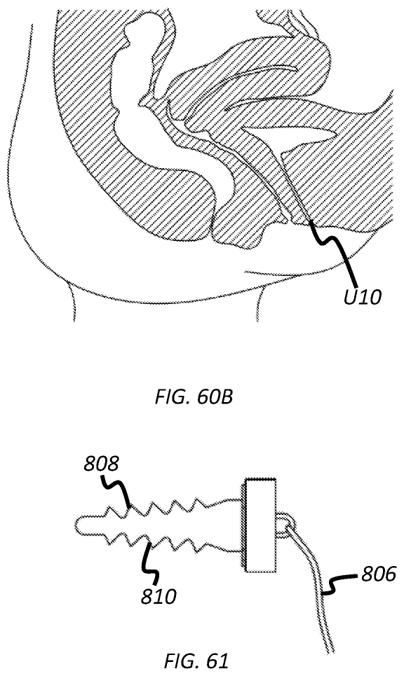

FIG. 60A shows an embodiment of the present invention to reduce the urine flow temporarily from the bladder by using a plug 800 and sealing the urethra U. The plug 800 may be placed into the urethral ostium U10 (see FIG. 60B) in a manner similar to the self-catheterization that individuals with the need for continuous urinary drainage undertake. Non-limiting examples of the materials to make the plug 800 may comprise soft, flexible, biocompatible materials such as latex or silicone. The plug 800 may comprise a tapered portion 802 similar to the tip of Foley or rubber catheters and a base 804. A loop of string or suture material 806 is attached to the base 804 of the plug 800 to make it simple to remove the plug 800 when necessary. In some embodiments, ribs 808 or an adhesive 810 (FIG. 61) may be added to the tapered portion 802 of the plug 800 to hold it into the urethral ostium U10 with the bladder pressure of the urine in the urethra.

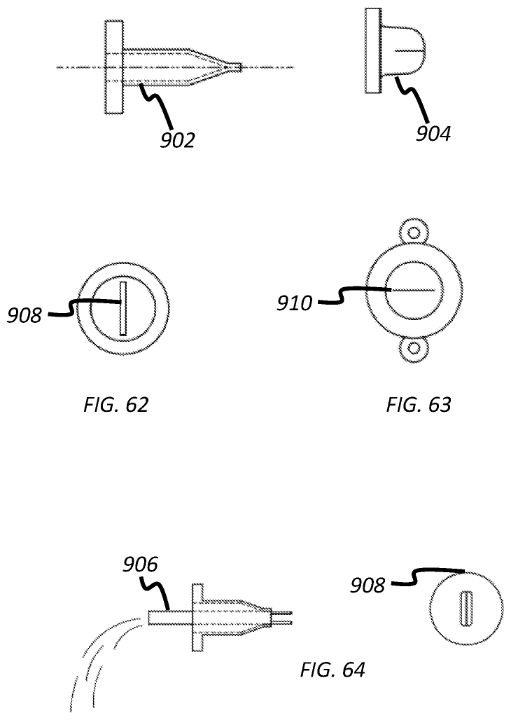

Referring to FIGS. 62-65, these figures show an embodiment of the present invention to reduce the urine flow from the bladder more permanently by sealing the ostium U10 with the valves 900 such as a duckbill valve 902 or dome valve 904. These valves operate as one-way valves that permit flow in only one direction. To open the duckbill valve 902, a hollow drainage tube 906 may be inserted inside the duckbill valve 902 to cause it to flex and thereby open up the sealing edge 908 of the duckbill valve 902 to allow urine to flow out of the bladder (FIG. 64). In some embodiments, the drainage tube 906 could be a disposable plastic, single use device or a stainless-steel reusable tube. To open the dome valve 904, a lateral pressure on the dome valve 904 by using a fingertip is necessary to cause it to flex and thereby open up the sealing edge 910 of the dome valve 904 to allow urine to flow out of the bladder (FIG. 65). In some embodiments, the valves are made of a soft, flexible, biocompatible material, such as, latex or silicone or other similar materials.

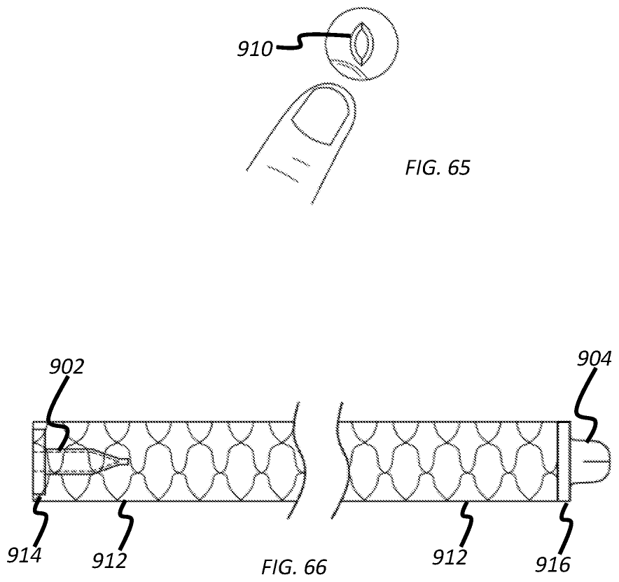

FIG. 66 shows means for stabilizing the duckbill valve 902 or the dome valve 904 into the urethra. The fluid pressure from the bladder exerted on the valves 902 and 904 might cause them to be dispelled. To mitigate this risk, a stent like configuration is proposed where a urethra stent 912 may be used as the delivery means for the valves 902 and 904. In some embodiments, the duckbill valve 902 may be located in the at the proximal end 914 of the stent 912. In some other embodiments, the dome valve 904 may be positioned at the distal end 916 of the stent 912. The opening of the valves 902 and 904 in FIG. 66 may be similar as described above for FIGS. 64 and 65. When the valves 902 and 904 are opened with lateral pressure, urine flows through the stent 912. In some embodiments, the stent configuration 912 may be further stabilized by the ingrowth of the tissue around the stent opening.

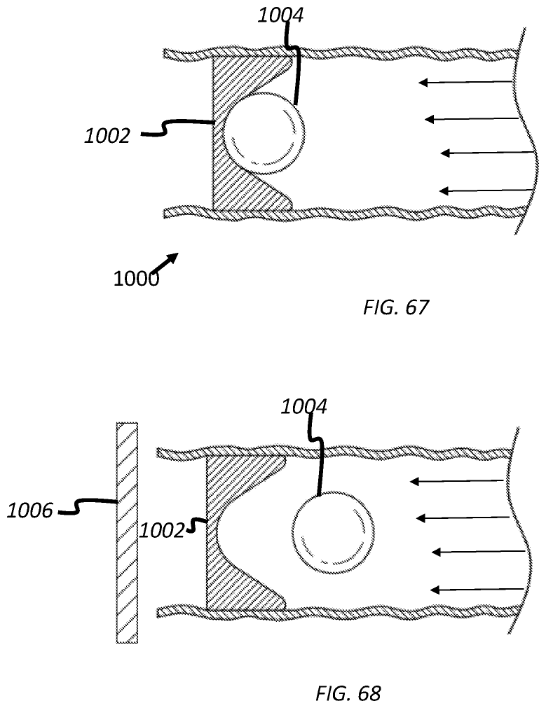

Referring to FIGS. 67, 68, these figures show another embodiment of the present invention for controlling the flow of urine by means of a magnetic valve housing 1000. The magnetic valve housing 1000 comprises a receiver 1002 which is shaped to mate with the associated magnetic sealing sphere 1004. The fluid pressure F from the bladder exerted on the magnetic valve housing 1000 in the urethra will keep it in the closed position, as shown in FIG. 67. When urination is desired (FIG. 68), the magnetic valve housing 1000 may be opened by placing an external magnet 1006 of the same polarity of the associated magnetic sealing sphere 1004 to repel the magnetic sealing sphere 1004. The same polarity of the external magnet 1006 and the magnetic sealing sphere 1004 will drive the sphere 1004 out of the receiver 1002 for opening the port to flow of urine. In some embodiments, the receiver 1002 may be made of a weaker magnate of the opposite polarity of the magnetic sealing sphere 1004. Once the bladder has been emptied, the external magnet 1006 is moved away from the receiver 1002 such that the opposite polarity of the weaker magnetic receiver 1002 attracts the magnetic sphere 1004 and the magnetic sealing sphere 1004 falls back into the receiver 1002 to close the magnetic valve housing 1000.

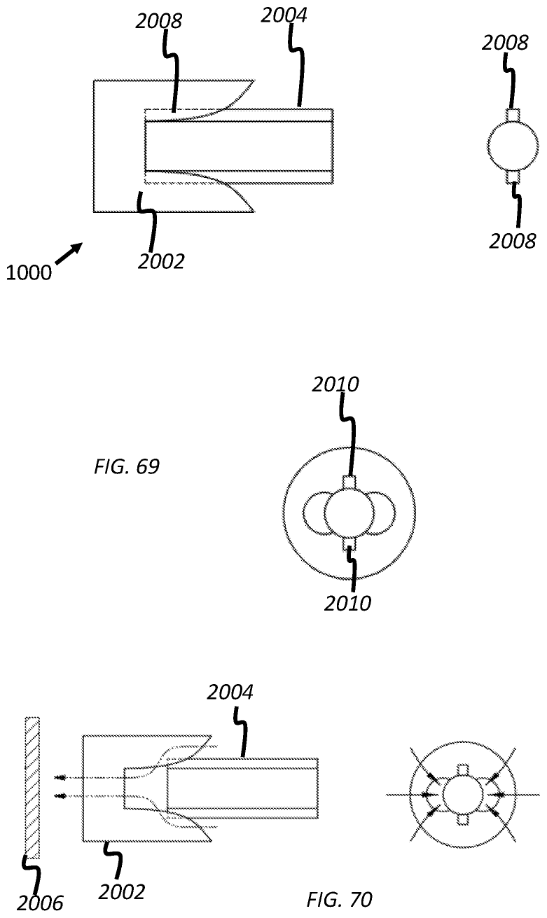

FIG. 69 shows another embodiment of the present invention for controlling the flow of urine by means of a magnetic valve housing 2000. The magnetic valve housing 2000 comprises a receiver 2002 which is shaped to mate with the associated magnetic rod-shaped member 2004 to stop the urine flow inside the magnetic valve housing 2000. In FIG. 69, the fluid pressure from the bladder exerted on the magnetic rod-shaped member 2004 will keep the magnetic valve housing 2000 in the closed position. When urination is desired (FIG. 70), the magnetic valve housing 2000 may be opened by placing an external magnet 2006 of the same polarity of the associated magnetic rod-shaped member 2004 to repel the magnetic rod-shaped member 2004. The same polarity of the external magnet 2006 and the magnetic rod-shaped member 2004 will drive the magnetic rod-shaped member 2004 out of the receiver 2002 for opening the channels 2008 to allow the flow of urine (FIG. 70). The magnetic rod-shaped member 2004 may comprise keys 2008 that mate with key slots 2010 to maintain the orientation of the two members 2008 and 2010. In some embodiments, the receiver 2002 may be made of a weaker magnate of the opposite polarity of the magnetic rod-shaped member 2004. Once the bladder has been emptied, the external magnet 2006 is moved away from the receiver 2002 such that the opposite polarity of the weaker magnetic receiver 2002 attracts the magnetic rod shaped member 2004 and the magnetic rod shaped member 2004 falls back into the receiver 2002 to close the magnetic valve housing 1000.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An apparatus useable to change a contour of a urethra comprising:
a body having a first configuration and a second configuration;
an anchoring mechanism usable to anchor said body at a location adjacent to a urethra;
wherein in said first configuration, said body allows urine to flow through said urethra;
wherein in said second configuration, said body changes shape such that said urethra is displaced from a natural position to a compressed position between said body and an anatomic structure of a patient, thereby reducing urine flow through said urethra.

2. The apparatus of claim 1, wherein said body comprises two sheets of materials bonded together.

3. The apparatus of claim 2, wherein said materials to form said body comprises polypropylene, polyethylene, nylon or PVC coated fabrics.

4. The apparatus of claim 2, wherein a bottom sheet of material is stiffer than a top sheet of material of said body so that said body expands unidirectionally towards a top.

5. The apparatus of claim 1, wherein in said second configuration, said body inflates with a fluid to change its shape.

6. The apparatus of claim 1, wherein said anchoring mechanism comprises:
a flexible tube with an access port at a proximal end of said body;
a suture at a distal end of said body,
an anchoring hook attached to a distal end of said suture;
wherein said anchoring hook engages tissues to anchor said body at said location adjacent to said urethra and said access port is secured at or above a skin level.

7. The apparatus of claim 6, wherein said flexible tube is configured to be sealed by inserting a one-way valve inside said access port.

8. The apparatus of claim 7, wherein said one-way valve comprises a duckbill valve, a dome valve, a flapper valve or a combination of a plug and an absorbable cap.

9. The apparatus of claim 8, wherein said combination of said plug and said absorbable cap are made of implantable, non-bio absorbing materials.

10. The apparatus of claim 6, wherein said anchoring mechanism further comprises an undulated pattern on a surface of said body to create friction at said location adjacent to said urethra.

11. The apparatus of claim 1, wherein said body contains an active agent and comprises a semi permeable membrane or an impermeable membrane having plurality of capillary paths on a surface of said body such that said active agent leaks out through said semi permeable membrane or said plurality of capillary paths of said impermeable membrane to promote cell growth at said location adjacent to said urethra.

12. The apparatus of claim 1, wherein said body comprises a piezoelectric actuator having a non-flexing base and a flexing stripe on said non-flexing base.

13. The apparatus of claim 12, wherein said flexing stripe expands to change said contour of said urethra.

14. The apparatus of claim 1, wherein said body comprises a spring jack having a flexible band attached to threaded collars.

15. The apparatus of claim 14, wherein said flexible band bends into a semi-circular orientation to change said contour of said urethra.

16. An apparatus configured to modulate urine flow from a urethra, comprising:
a tissue grasping device having a distal grasping end and a proximal grasping end connected by a filament;
wherein said distal grasping end anchors tissues adjacent to a urethra and said proximal grasping end anchors tissues adjacent to a deployment site, thereby compressing said urethra and modulating urine flow through said urethra.

17. The apparatus of claim 16, wherein said distal grasping end comprises forward-facing barbs and said proximal grasping end comprises reverse facing barbs.

18. The apparatus of claim 16, wherein a plurality of tissue grasping devices are used to modulate said urine flow through said urethra.

19. The apparatus of claim 16, wherein said deployment site comprises uterofacial tissue or vaginal wall.

20. A method of inhibiting urine flow through a urethra comprising:
positioning a device at a desired location adjacent to said urethra in a first configuration;
changing a shape of said device to a second configuration thereby
compressing said urethra against adjacent tissue and inhibiting said urine flow through said urethra.

21. The method of claim 20, wherein said step of positioning said device at said desired location adjacent to said urethra comprises engaging an anchoring hook of said device by an angulated needle of an introducer and inserting said device adjacent to said urethra through an abdominal incision.

22. The method of claim 20, wherein said step of changing said shape of said device to said second configuration comprises inflating said device by a fluid.

23. The method of claim 20, wherein said step of changing said shape of said device to said second configuration comprises flexing a flexible member of said device.

24. The method of claim 20, wherein said step of positioning said device at said desired location adjacent to said urethra comprises anchoring tissues adjacent to said urethra through a distal grasping end of said device and anchoring tissues adjacent to a deployment site of said device through a proximal grasping end.

25. The method of claim 20, wherein inhibiting urine flow through said urethra further comprises inserting a one-way valve into a urethral ostium and reducing urine flow temporarily by sealing said urethra.

26. The method of claim 20, wherein inhibiting urine flow through said urethra further comprises inserting a magnetic valve housing into said urethra.

27. The method of claim 26, wherein inhibiting urine flow by inserting by magnetic valve housing further comprises:
providing a magnetic sphere shaped to match an optionally magnetic receiver of opposite polarity inside said magnetic valve housing; and
inhibiting flow of urine by placing said magnetic sphere inside said magnetic receiver.

28. The method of claim 26, wherein inhibiting urine flow by inserting by magnetic valve housing further comprises:
providing a magnetic rod shaped to match an optionally magnetic receiver of opposite polarity inside said magnetic valve housing; and
inhibiting flow of urine by placing said magnetic rod inside said magnetic receiver.
